(19) United States
(12) Patent Application Publication
(10) Pub. No.: US 2026/0259189 A1
(43) Pub. Date: Sep. 3, 2026

(54) SYSTEM AND METHOD FOR TESTING A PAINTED SURFACE OF A STRUCTURE

(71) Applicant: THE BOEING COMPANY, Arlington, VA (US)

(72) Inventor: Kellon Darwin Johnson, Granite Falls, WA (US)

(73) Assignee: THE BOEING COMPANY, (omitted)

(21) Appl. No.: 19/656,423

(22) Filed: Apr. 23, 2026

Related U.S. Application Data
(63) Continuation-in-part of application No. 18/628,795, filed on Apr. 7, 2024.

(51) Int. Cl. G01N 33/32; G01N 1/36; G01N 17/00; G01N 19/04

(52) U.S. Cl. CPC G01N 33/32; G01N 1/36; G01N 17/00; G01N 19/04

(57) ABSTRACT

A system and a method or testing a painted surface of a structure include a frame, and a window coupled to the frame. An internal chamber is defined between the frame and the window. The internal chamber is configured to receive a testing medium and sealingly secure the testing medium against the painted surface.

### CROSS-REFERENCE TO RELATED APPLICATIONS

This application is a continuation-in-part of U.S. Patent Application No. 18/628,795, filed April 7, 2024, entitled “Finish Saturator for Enhanced Paint Finish Testing,” which is hereby incorporated by reference in its entirety.

### FIELD OF THE DISCLOSURE

Examples of the present disclosure generally relate to a system and a method for testing a painted surface of a structure, such as a wall, ceiling, floor, portion of a vehicle (such as a fuselage or walls within an internal cabin), or the like.

### BACKGROUND OF THE DISCLOSURE

Various structures have surfaces that are painted. For example, walls, ceilings, floors, interior and exterior portions of vehicles, and the like can have painted surfaces.

In certain industries, such as the automotive and aerospace industries, paint finishes of structures are tested in relation to adhesion and weather resistance to ensure the longevity and durability of the painted surfaces. Known testing methods include manually-assembled material, such as makeshift bags formed of plastic and duct tape.

However, it has been found that such testing methods can lead to inconsistent test conditions and unreliable results.

### SUMMARY OF THE DISCLOSURE

A need exists for a standardized, efficient, effective, and dependable paint testing system and method to enhance quality control and product durability.

### BRIEF DESCRIPTION OF THE DRAWINGS

FIG. 1 illustrates a front view of a system for testing a painted surface of a structure, according to an example of the present disclosure.

FIG. 2 illustrates a rear view of the system of FIG. 1.

FIG. 3 illustrates an isometric rear view of the system of FIG. 1.

FIG. 4 illustrates a top view of the system of FIG. 1.

FIG. 5 illustrates a first side view of the system of FIG. 1.

FIG. 6 illustrates a second side view of the system of FIG. 1.

FIG. 7 illustrates an isometric rear view of the system, according to an example of the present disclosure.

FIG. 8 illustrates an isometric rear view of the system having a protective film being removed.

FIG. 9 illustrates an isometric front view of the system separated from a surface of a structure, according to an example of the present disclosure.

FIG. 10 illustrates an isometric front view of the system coupled to the surface of the structure, according to an example of the present disclosure.

FIG. 11 illustrates a front view of the system retaining a testing medium on the surface of the structure.

FIG. 12 illustrates a flow chart of a method, according to an example of the present disclosure.

### DETAILED DESCRIPTION OF THE DISCLOSURE

The foregoing summary, as well as the following detailed description of certain embodiments will be better understood when read in conjunction with the appended drawings. As used herein, an element or step recited in the singular and preceded by the word "a" or "an" should be understood as not necessarily excluding the plural of the elements or steps. Further, references to "one embodiment" are not intended to be interpreted as excluding the existence of additional embodiments that also incorporate the recited features. Moreover, unless explicitly stated to the contrary, embodiments "comprising" or "having" an element or a plurality of elements having a particular condition can include additional elements not having that condition.

Examples of the present disclosure provide systems and methods that are configured to assess weather resistance and adhesion properties of paint finishes of structures, such as used in relation to vehicles. The systems and methods provide a finish saturator for testing weather resistance and adhesion properties of paint finishes.

The systems and methods described herein provide a standardized, user-friendly device aimed at testing the adhesion and weather resistance of paint finishes. In at least one example, the systems and methods include a frame, such as a rectangular, square, circular or other such shape frame, which can be formed of a plastic, such as polyurethane. The frame can include a pre-applied, water-resistant, non-residual adhesive along a rear perimeter surface. The frame defines an internal chamber, such as a central pocket, configured to secure a testing medium (such as a water-soaked cloth, sponge, or the like) against a surface having a painted finish, thereby providing a standardized, consistent, reliable, and efficient testing system and method for testing adhesion and weather resistance of the painted surface.

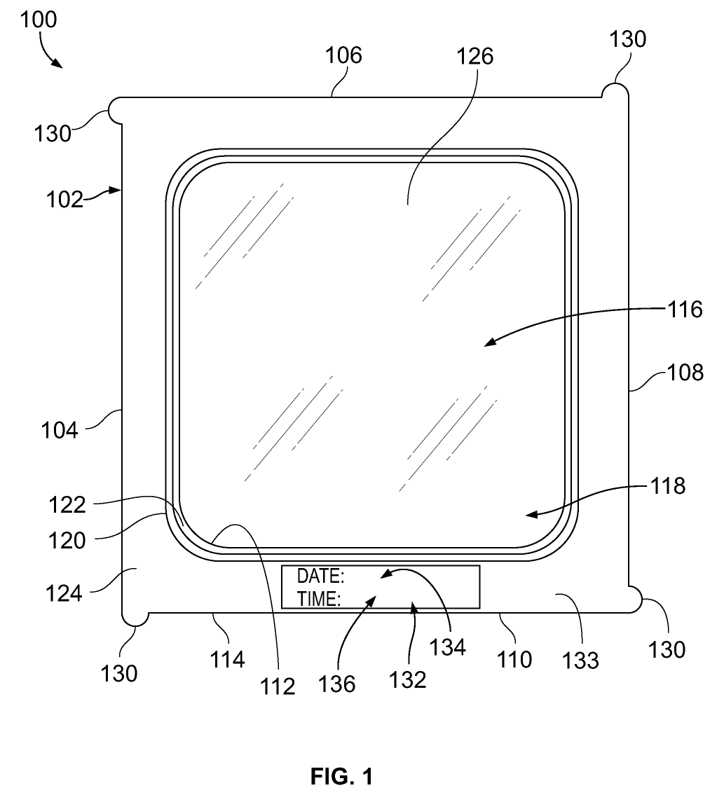

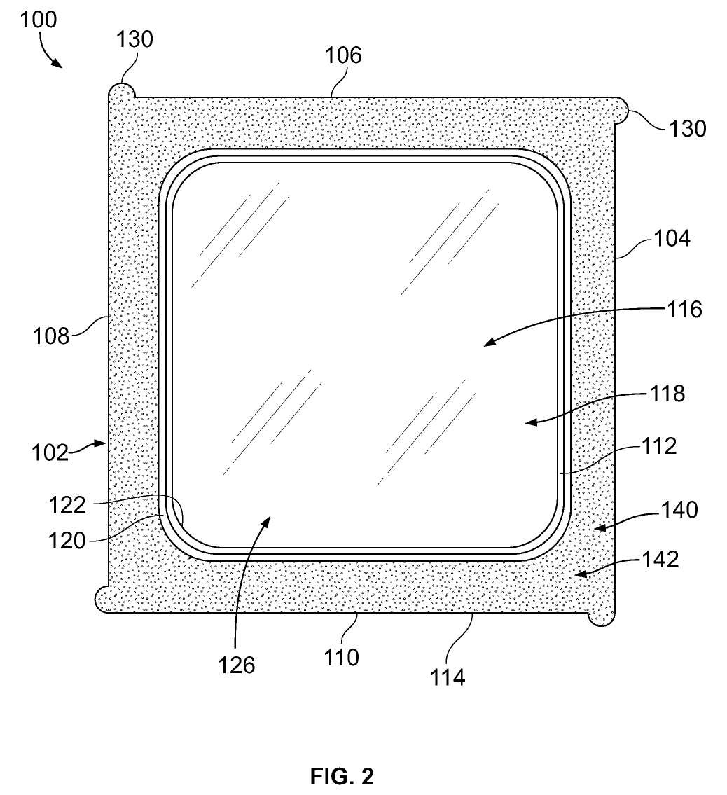

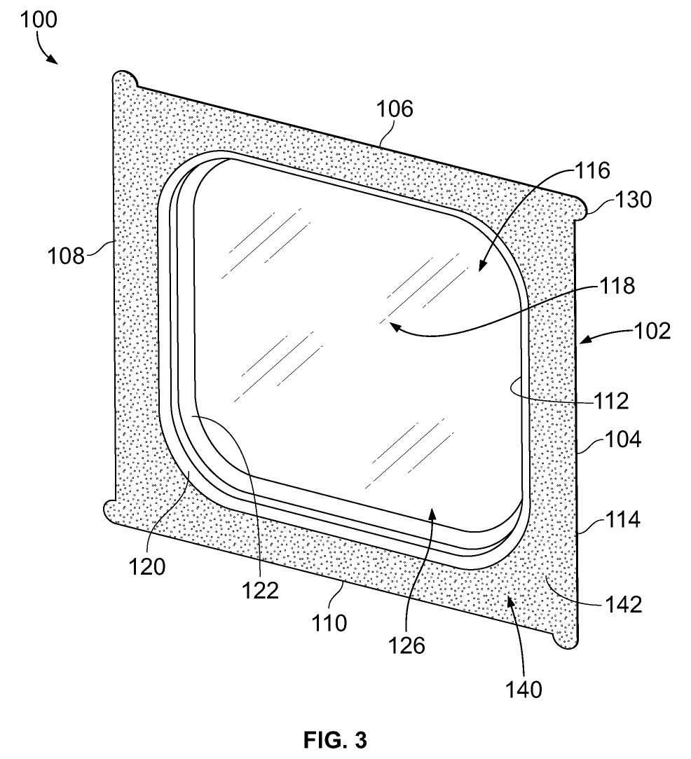

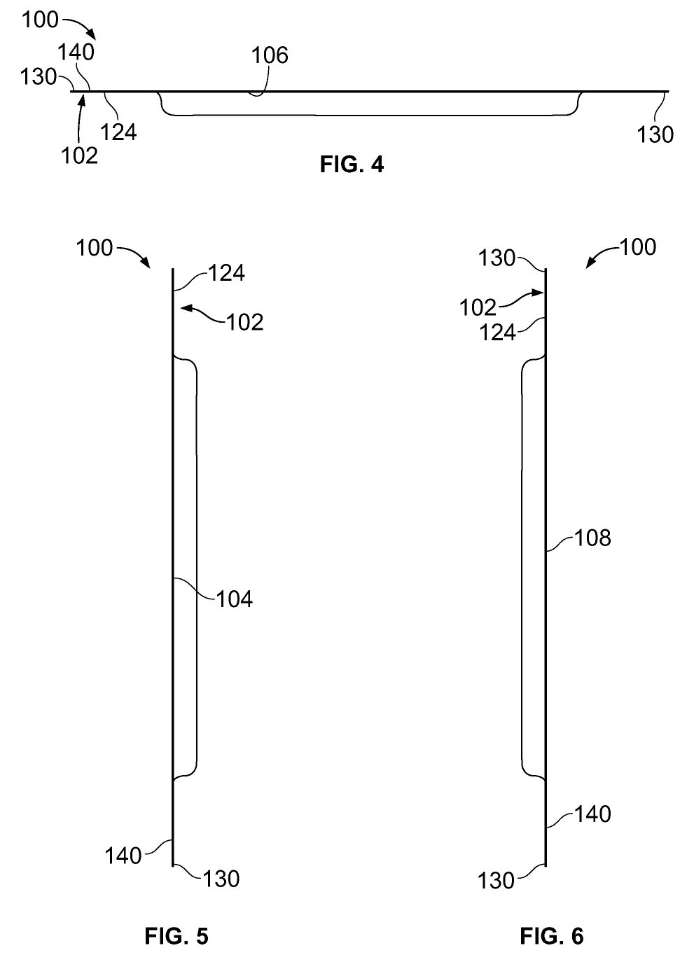

FIG. 1 illustrates a front view of a system 100 for testing a painted surface of a structure, according to an example of the present disclosure. FIG. 2 illustrates a rear view of the system 100 of FIG. 1. FIG. 3 illustrates an isometric rear view of the system 100 of FIG. 1. FIG. 4 illustrates a top view of the system 100 of FIG. 1. FIG. 5 illustrates a first side view of the system 100 of FIG. 1. FIG. 6 illustrates a second side view of the system 100 of FIG. 1. Referring to FIGS. 1-6, in at least one example, the system 100 provides a finish saturator.

The system 100 includes a frame 102 configured to be applied to the painted surface. As shown, the frame 102 can be shaped as a rectangle or square, and having a first beam 104, a second beam 106, a third beam 108 opposite from the first beam 104, and a fourth beam 110 opposite from the second beam 106. Optionally, the frame 102 can be shaped differently than shown. For example, the frame 102 can be shaped as a circle, triangle, etc. The frame 102 includes an inner perimeter 112 and an outer perimeter 114 defined along the first beam 104, the second beam 106, the third beam 108, and the fourth beam 110. A internal chamber 116 is defined by the inner perimeter 112 of the frame 102. A window 118 spans across the internal chamber 116 from the inner perimeter 112. The window 118 can be integrally formed with the frame 102. Optionally, the window 118 can be a different piece that is connected to the frame 102.

In at least one example, the window 118 is formed of the same material as the frame 102, but can be transparent to allow viewing therethrough. For example, the transparent window 118 allows viewing of a testing medium retained within the internal chamber 116. The frame 102 can also be transparent. As another example, instead of a clear window, an opaque or semi-opaque cover can span across the internal chamber 116.

In at least one example, the inner perimeter 112 of the frame 102 includes one or more walls outwardly extending from a base surface 124 of the frame 102. For example, a first stepped wall 120 extends from an interior edge of the base surface 124, and a second stepped wall 122 extends from an interior edge of the first stepped wall 120. The first stepped wall 120 provides a first stepped height above the base surface 124 of the frame 102, while the second stepped wall 122 provide a second stepped height above the first stepped wall 120. In this manner, an interior pocket 126 is formed between the inner perimeter 112 (such as by the first stepped wall 120 and the second stepped wall 122) and a lower surface of the window 118. Optionally, the frame 102 can include more or fewer stepped walls than shown. For example, a single large wall can outwardly extend from the inner perimeter 112. As another example, three or more stepped walls can outwardly extend from the inner perimeter. In at least one other example, walls may not outwardly extend from the inner perimeter 112. Instead, the window 118 can be coplanar with the base surface 124 (before a testing medium is disposed underneath the window 118).

As shown, one or more tabs 130 outwardly extend from the outer perimeter 114. The tabs 130 can be semi-circular protrusions. As shown, tabs 130 are located at terminal ends of junctions (a) the first beam 104 and the second beam 106, (b) the second beam 106 and the third beam 108, (c) the third beam 108 and the fourth beam 110, and the fourth beam 110 and the first beam 104. The tabs 130 are configured to allow an individual to grasp and peel the frame 102 away from a surface on which the system 100 is applied. Optionally, the frame 102 can include more or fewer tabs 130 than shown. For example, the frame 102 can include one tab 130. Alternatively, the frame 102 may not include any tab.

In at least one example, the frame 102 also includes a test information indicator 132. As shown, the test information indicator 132 includes text and/or graphics shown on a front surface 133 of the frame 102, such as on the fourth beam 110. Optionally, the test information indicator 132 can be located on other portions of the frame 102. In at least one example, the test information indicator 132 includes a location for a date 134 and a time 136 of a test. By indicating the date 134 and the time 136 of a test on the test information indicator 132, an individual is able to readily determine whether a test of a paint surface has ended or should still proceed. Alternatively, the frame 102 may not include the test information indicator.

A rear surface 140 of the frame 102 includes an adhesive 142. In at least one example, the adhesive 142 is applied over an entirety of the rear surface 140. The adhesive 142 can include a glue, two-sided tape, and/or the like, which allows the frame 102 to sealingly and removably secure to a surface of a structure.

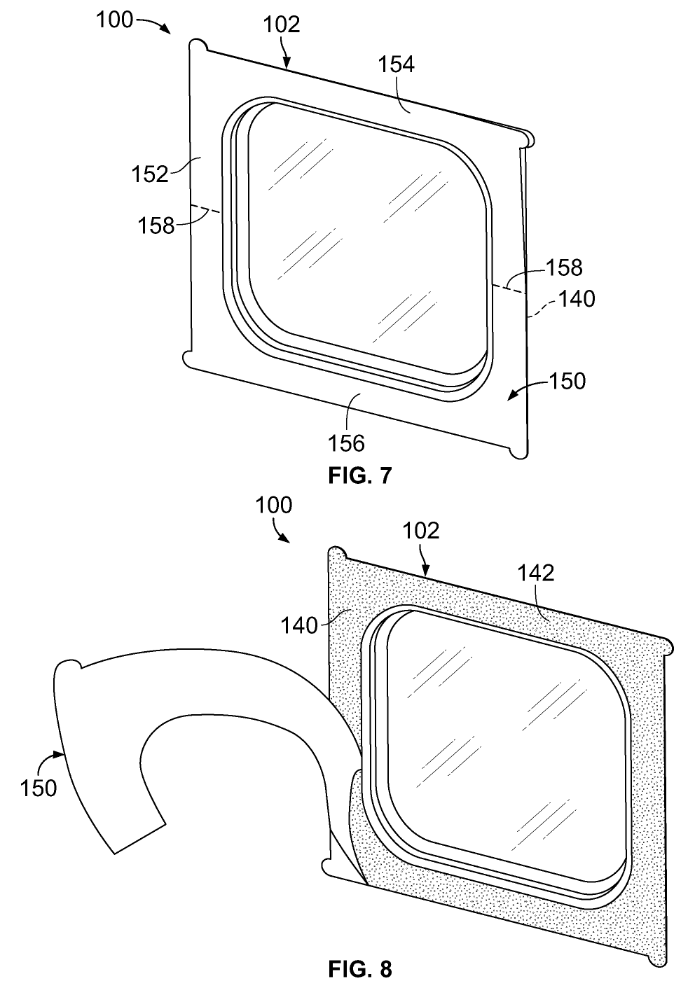

FIG. 7 illustrates an isometric rear view of the system 100, according to an example of the present disclosure. In at least one example, a protective film 150, such as formed of a flexible plastic, is disposed on the rear surface 140 of the frame 102. The protective film 150 covers the adhesive 142 and ensures that the system 100 does not inadvertently secure to a structure or object. That is, the protective film 150 clings to the adhesive 142 of the rear surface 140, but provides a non-adhesive outer surface 152. As shown, the protective film 150 can include a first half 154 and a second half 156 separated at seams 158. The seams 158 allow an individual to easily grasp an edge of the protective film 150 and peel the protective film 150 off the rear surface 140 of the frame 102. Optionally, the protective film 150 may not include separable halves, but instead be a unitary structure that can be peeled off via interior or exterior edges.

FIG. 8 illustrates an isometric rear view of the system 100 having the protective film 150 being removed away from the rear surface 140. When the protective film 150 is removed, the adhesive 142 on the rear surface 140 is exposed. The adhesive 142, as exposed, can then be used to secure the frame 102 and therefore the system 100 to a surface of a structure.

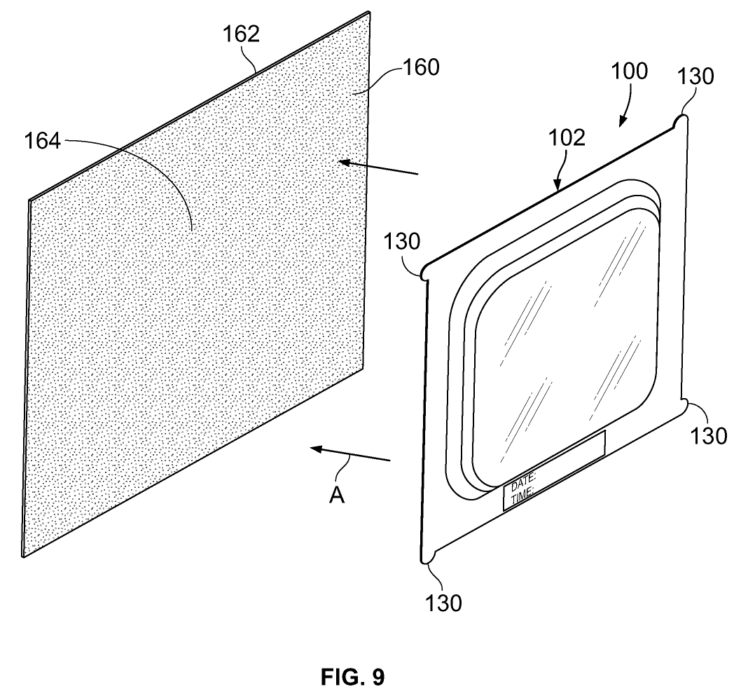

FIG. 9 illustrates an isometric front view of the system 100 separated from a surface 160 of a structure 162, according to an example of the present disclosure. In at least one example, the surface 160 includes paint 164. The structure 162 can be a portion of a vehicle, such as an exterior or interior surface of the vehicle. The system 100 is used to test the paint 164. In particular, the system 100 is used to test the adhesion and weather resistance of the paint 164.

In order to test the paint 164, the system 100 is urged toward and onto the surface 160 in the direction of arrow A. Referring to FIGS. 1-9, the adhesive 142 on the rear surface 140 of the frame 102 sealingly engages the surface 160, thereby providing a liquid-tight seal therewith. An individual can peel the system 100 away from the surface 160 via the tabs 130, and re-apply the system 100 to the surface 160, as desired.

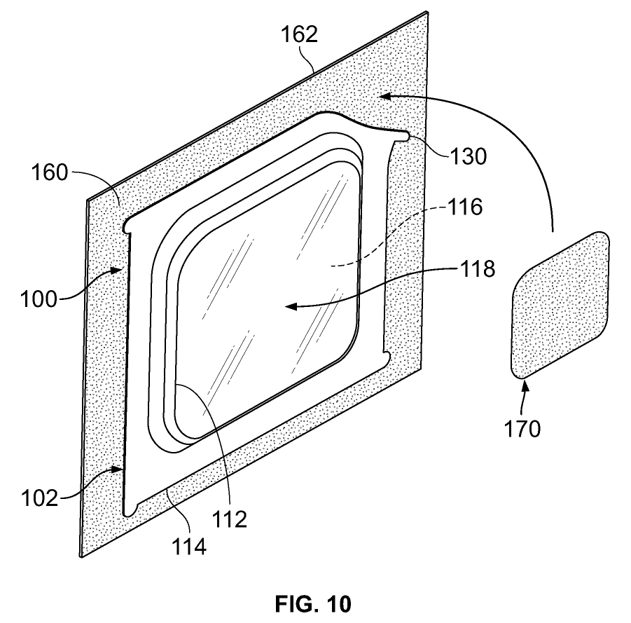

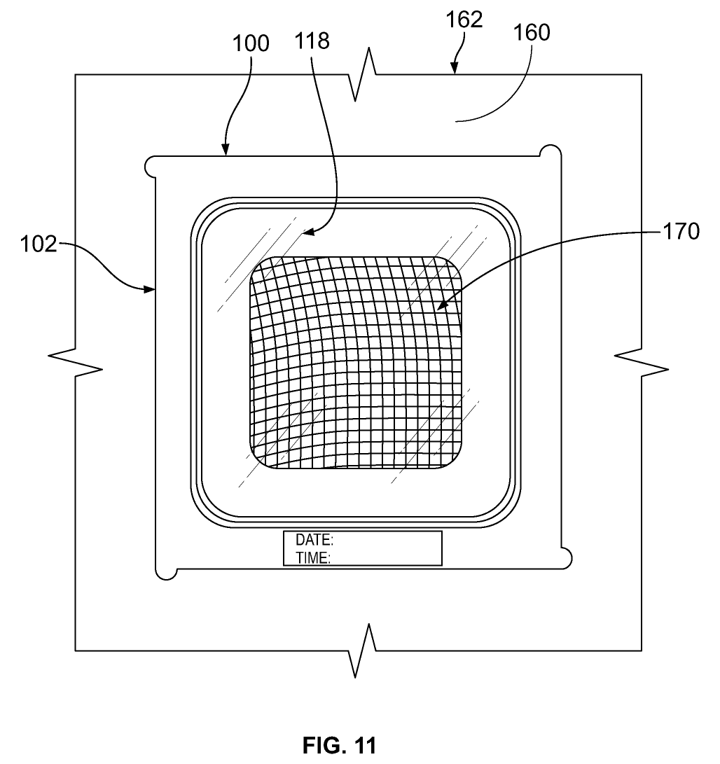

FIG. 10 illustrates an isometric front view of the system 100 coupled to the surface 160 of the structure 162, according to an example of the present disclosure. FIG. 11 illustrates a front view of the system 100 retaining a testing medium 170 on the surface of the structure. Referring to FIGS. 10 and 11, when the system 100 is secured to the surface 160, a tab 130 can be pulled to temporarily peel the frame 102 away from the surface 160. Then, the testing medium 170 can be disposed within the internal chamber 116 underneath the window 118, and the frame 102 can be reapplied to the surface 160 (as shown in FIG. 11) to ensure that the testing medium 170 is sealed within the internal chamber against the surface 160 of the structure 162. As such, the testing medium 170 is trapped between the surface 160 of the structure 162, an underside of the window 118, and the inner perimeter 112 of the frame 102. The window 118 can exert a compressive force into the testing medium 170, thereby ensuring that the testing medium 170 remains in contact with the surface 160. The system 100 ensures that the testing medium 170 remains within the internal chamber 116, and contacting the surface 160, while the frame 102 and the window 118 prevent liquid from passing out of the internal chamber 116.

In at least one example, the testing medium 170 is a saturated cloth, sponge, pad, or the like. For example, the testing medium 170 can be a cloth or sponge saturated with water.

Referring to FIGS. 1-11, the system 100 is configured for testing a painted surface 160 of a structure 162. The system 100 includes the frame 102 and the window 118 coupled to the frame 102. In at least one example, the window 118 is integrally formed with the frame 102. For example, the frame 102 and the window 118 can be integrally molded and formed as a single, unitary piece. Optionally, the window 118 can be a separate component that is separately connected to the frame 102. The internal chamber 116 is defined between the frame 102 and the window 118. The internal chamber 116 is configured to receive the testing medium 170 and sealingly secure the testing medium 170 against the painted surface 160.

In at least one example, the frame 102 can have an area of 36 square inches (6 inches X 6 inches), and can have a thickness of 3 millimeters (mm). The internal chamber 116 can have an area of 25 square inches, and can have a depth of 0.20 inches. Optionally, the frame 102 can be larger or smaller than 36 square inches (and the internal chamber 116 can have an area that is greater or less than 25 square inches with a depth that is greater or less than 0.20 inches), and the thickness can be greater or less than 3 mm. In at least one example, the frame 102 is formed of polyurethane. Optionally, the frame 102 can be formed of another flexible material, such as rubber.

In at least one example, the adhesive 142 is disposed on an entirety of the rear surface 140 of the frame 102, such as on rear surfaces of the beams 104, 106, 108, and 110. In at least one example, the adhesive 142 is water-resistant and leaves no residue.

As shown and described the system 100 allows for consistent testing for adhesion and weather resistance of the paint 164 on the surface 160 of the structure 162. The system 100 is easy to use, and provides a simplified, yet consistent, setup process due to the pre-applied adhesive 142 and designated internal chamber 116 configured to receive and retain the testing medium 170. The system 100 provides uniform testing conditions, and leads to reliable testing results. Moreover, the system 100 improves testing efficiency, and reduces testing preparation time, as there is no need to construct an improvised device with duct tape, for example.

The systems and methods described herein are particularly useful for industries which value the integrity and endurance of paint finishes, such as in relation to vehicles (for example, commercial aircraft, automobiles, and the like). Examples of the present disclosure provide consistent, reliable, effective, and efficient systems and methods for testing paint finishes of structures, improve quality control. and augment research and development.

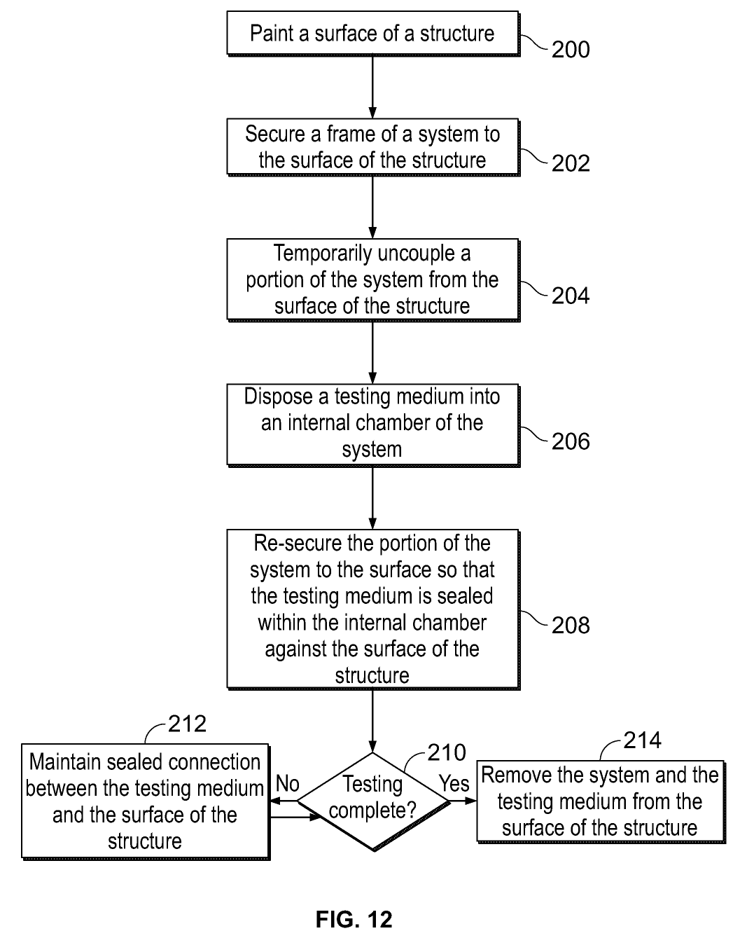

FIG. 12 illustrates a flow chart of a method, according to an example of the present disclosure. Referring to FIGS. 1-12, at 200, a surface 160 of a structure 162 is painted, thereby providing a painted surface to be tested. At 202, the frame 102 of the system 100 is secured to the surface 160 of the structure 162. At 204, a portion of the system 100 is temporarily uncoupled (such as by peeling away, via a tab 130) from the surface 160 of the structure, thereby providing an opening into an internal chamber 116 of the system 100. At 206, the testing medium 170 is then disposed within the internal chamber 116. At 208, the portion of the system 100 is then re-secured to the surface 160 (via the adhesive 142 on the rear surface 140 of the frame 102) so that the testing medium 170 is sealed within the internal chamber 116 against the surface 160 of the structure 162.

At 210, it is determined if testing is complete. For example, a test can last a predetermined period of time, such as twenty-four hours, in which the saturated testing medium 170 is disposed against the surface 162 to determine if the painted finish can withstand contact with the liquid. After the predetermined period of time, the properties of the painted finish can then be assessed.

If testing is not complete at 210, the method proceeds to 212, at which the sealed connection between the testing medium 170 and the surface 160 is maintained. The method then returns to 210.

If testing is complete at 210 (such as after a predetermined period of time for a test), the method proceeds to 214, at which the system 100 and the testing medium 170 are removed from the surface 160 of the structure 162. The surface 160 can then be assessed.

Further, the disclosure includes examples according to the following clauses:

Clause 1. A system for testing a painted surface of a structure, the system comprising:

a frame; and

a window coupled to the frame, wherein an internal chamber is defined between the frame and the window, and wherein the internal chamber is configured to receive a testing medium and sealingly secure the testing medium against the painted surface.

Clause 2. The system of Clause 1, wherein the internal chamber is defined between an inner perimeter of the frame and the window.

Clause 3. The system of Clause 2, wherein the inner perimeter includes one or more walls outwardly extending from a base surface of the frame.

Clause 4. The system of Clause 3, wherein the one or more walls comprises a first stepped wall and a second stepped wall.

Clause 5. The system of any of Clauses 1-4, wherein the window is transparent and allows viewing of the testing medium within the internal chamber.

Clause 6. The system of any of Clauses 1-5, further comprising one or more tabs outwardly extending from an outer perimeter of the frame.

Clause 7. The system of any of Clauses 1-6, further comprising a test information indicator on a front surface of the frame.

Clause 8. The system of Clause 7, wherein the test information indicator comprises a first location for a date of a test, and a second location for a time of the test.

Clause 9. The system of any of Clauses 1-8, wherein a rear surface of the frame comprises an adhesive configured to sealingly and removably secure the system to the painted surface of the structure.

Clause 10. The system of Clause 9, further comprising a protective film disposed on the rear surface of the frame, wherein the protective film is configured to be removed before the system is secured to the painted surface.

Clause 11. The system of any of Clauses 1-10, wherein the testing medium comprises a saturated cloth.

Clause 12. The system of any of Clauses 1-11, wherein the frame is formed of polyurethane.

Clause 13. A method for a system for testing a painted surface of a structure, the system comprising:

a frame; and

a window coupled to the frame, wherein an internal chamber is defined between the frame and the window, wherein the internal chamber is configured to receive a testing medium and sealingly secure the testing medium against the painted surface, and wherein the testing medium comprises a saturated cloth,

the method comprising:

receiving the testing medium within the internal chamber of the system; and

sealingly securing the testing medium against the painted surface for a predetermined period of time.

Clause 14. The method of Clause 13, wherein the internal chamber is defined between an inner perimeter of the frame and the window.

Clause 15. The method of Clauses 13 or 14, further comprising allowing the testing medium to be viewed through the window.

Clause 16. The method of any of Clauses 13-15, further comprising grasping one or more tabs outwardly extending from an outer perimeter of the frame to remove the system from the painted surface.

Clause 17. The method of any of Clauses 13-16, further comprising providing a date of a test and a time of the test on a test information indicator on a front surface of the frame.

Clause 18. The method of any of Clauses 13-17, wherein said sealingly securing comprises using adhesive on a rear surface of the frame to sealingly and removably secure the system to the painted surface of the structure.

Clause 19. The method of Clause 18, further comprising removing a protective film disposed on the rear surface of the frame before the system is secured to the painted surface.

Clause 20. A system for testing a painted surface of a structure, the system comprising:

a testing medium comprising a saturated cloth;

a frame formed of polyurethane, wherein the frame comprises an inner perimeter and an outer perimeter, wherein one or more tabs outwardly extend from the outer perimeter, and wherein a rear surface of the frame comprises an adhesive configured to sealingly and removably secure the system to the painted surface of the structure;

a protective film disposed on the rear surface of the frame, wherein the protective film is configured to be removed before the system is secured to the painted surface;

a test information indicator on a front surface of the frame, wherein the test information indicator comprises a first location for a date of a test, and a second location for a time of the test; and

a window coupled to the frame, wherein an internal chamber is defined between the inner perimeter of the frame and the window, wherein the internal chamber receives the testing medium and sealingly secures the testing medium against the painted surface, wherein the window is transparent and allows viewing of the testing medium within the internal chamber.

As described herein, examples of the present disclosure provide standardized, efficient, effective, and dependable paint testing systems and methods that enhance quality control and product durability.

While various spatial and directional terms, such as top, bottom, lower, mid, lateral, horizontal, vertical, front and the like can be used to describe embodiments of the present disclosure, it is understood that such terms are merely used with respect to the orientations shown in the drawings. The orientations can be inverted, rotated, or otherwise changed, such that an upper portion is a lower portion, and vice versa, horizontal becomes vertical, and the like.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

It is to be understood that the above description is intended to be illustrative, and not restrictive. For example, the above-described embodiments (and/or aspects thereof) can be used in combination with each other. In addition, many modifications can be made to adapt a particular situation or material to the teachings of the various embodiments of the disclosure without departing from their scope. While the dimensions and types of materials described herein are intended to define the parameters of the various embodiments of the disclosure, the embodiments are by no means limiting and are exemplary embodiments. Many other embodiments will be apparent to those of skill in the art upon reviewing the above description. The scope of the various embodiments of the disclosure should, therefore, be determined with reference to the appended claims, along with the full scope of equivalents to which such claims are entitled. In the appended claims and the detailed description herein, the terms "including" and "in which" are used as the plain-English equivalents of the respective terms "comprising" and "wherein." Moreover, the terms "first," "second," and "third," etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the various embodiments of the disclosure, including the best mode, and also to enable any person skilled in the art to practice the various embodiments of the disclosure, including making and using any devices or systems and performing any incorporated methods. The patentable scope of the various embodiments of the disclosure is defined by the claims, and can include other examples that occur to those skilled in the art. Such other examples are intended to be within the scope of the claims if the examples have structural elements that do not differ from the literal language of the claims, or if the examples include equivalent structural elements with insubstantial differences from the literal language of the claims.

What is claimed is:

1. A system for testing a painted surface of a structure, the system comprising:
a frame; and
a window coupled to the frame, wherein an internal chamber is defined between the frame and the window, and wherein the internal chamber is configured to receive a testing medium and sealingly secure the testing medium against the painted surface.

2. The system of claim 1, wherein the internal chamber is defined between an inner perimeter of the frame and the window.

3. The system of claim 2, wherein the inner perimeter includes one or more walls outwardly extending from a base surface of the frame.

4. The system of claim 3, wherein the one or more walls comprises a first stepped wall and a second stepped wall.

5. The system of claim 1, wherein the window is transparent and allows viewing of the testing medium within the internal chamber.

6. The system of claim 1, further comprising one or more tabs outwardly extending from an outer perimeter of the frame.

7. The system of claim 1, further comprising a test information indicator on a front surface of the frame.

8. The system of claim 7, wherein the test information indicator comprises a first location for a date of a test, and a second location for a time of the test.

9. The system of claim 1, wherein a rear surface of the frame comprises an adhesive configured to sealingly and removably secure the system to the painted surface of the structure.

10. The system of claim 9, further comprising a protective film disposed on the rear surface of the frame, wherein the protective film is configured to be removed before the system is secured to the painted surface.

11. The system of claim 1, wherein the testing medium comprises a saturated cloth.

12. The system of claim 1, wherein the frame is formed of polyurethane.

13. A method for a system for testing a painted surface of a structure, the system comprising:
a frame; and
a window coupled to the frame, wherein an internal chamber is defined between the frame and the window, wherein the internal chamber is configured to receive a testing medium and sealingly secure the testing medium against the painted surface, and wherein the testing medium comprises a saturated cloth,
the method comprising:
receiving the testing medium within the internal chamber of the system; and
sealingly securing the testing medium against the painted surface for a predetermined period of time.

14. The method of claim 13, wherein the internal chamber is defined between an inner perimeter of the frame and the window.

15. The method of claim 13, further comprising allowing the testing medium to be viewed through the window.

16. The method of claim 13, further comprising grasping one or more tabs outwardly extending from an outer perimeter of the frame to remove the system from the painted surface.

17. The method of claim 13, further comprising providing a date of a test and a time of the test on a test information indicator on a front surface of the frame.

18. The method of claim 13, wherein said sealingly securing comprises using adhesive on a rear surface of the frame to sealingly and removably secure the system to the painted surface of the structure.

19. The method of claim 18, further comprising removing a protective film disposed on the rear surface of the frame before the system is secured to the painted surface.

20. A system for testing a painted surface of a structure, the system comprising:
a testing medium comprising a saturated cloth;
a frame formed of polyurethane, wherein the frame comprises an inner perimeter and an outer perimeter, wherein one or more tabs outwardly extend from the outer perimeter, and wherein a rear surface of the frame comprises an adhesive configured to sealingly and removably secure the system to the painted surface of the structure;
a protective film disposed on the rear surface of the frame, wherein the protective film is configured to be removed before the system is secured to the painted surface;
a test information indicator on a front surface of the frame, wherein the test information indicator comprises a first location for a date of a test, and a second location for a time of the test; and
a window coupled to the frame, wherein an internal chamber is defined between the inner perimeter of the frame and the window, wherein the internal chamber receives the testing medium and sealingly secures the testing medium against the painted surface, wherein the window is transparent and allows viewing of the testing medium within the internal chamber.
